(19) United States
(12) Patent Application Publication
(10) Pub. No.: US 2026/0240410 A1
(43) Pub. Date: Aug. 20, 2026

(54) ENDOSCOPY SUPPORT DEVICE, ENDOSCOPY SUPPORT METHOD, AND RECORDING MEDIUM

(71) Applicant: NEC Corporation, Tokyo (JP)

(72) Inventors: Takayuki OKUNO, Tokyo (JP); Naoto MAEDA, Tokyo (JP); Yuko MAEKAWA, Tokyo (JP); Takuma IGARASHI, Tokyo (JP); Hiroyasu SAIGA, Tokyo (JP); Yuji IWADATE, Tokyo (JP)

(73) Assignee: NEC Corporation, Tokyo (JP)

(21) Appl. No.: 19/161,343

(22) PCT Filed: Mar. 8, 2023
(86) PCT No.: PCT/JP2023/008903

§ 371 Date: Sep. 2, 2025

(51) Int. Cl. A61B 1/00; A61B 1/31; A61B 5/06; G06T 7/00

(52) U.S. Cl. CPC A61B 1/000094; A61B 1/000096; A61B 1/0002; A61B 1/00045; A61B 1/31; A61B 5/065; G06T 7/0012; G06T 2207/10068; G06T 2207/20081; G06T 2207/30028; G06T 2207/30096

(57) ABSTRACT

In an endoscopy support device, an image acquisition means for acquiring an endoscopic image captured by an endoscope. An identification information acquisition means for acquiring identification information of a patient. A position estimation means for estimating a position of the endoscope in a large intestine. A storage means for storing previous examination information associating the identification information of the patient, a position where a lesion was detected in a previous examination, an image of the lesion, and information regarding the lesion. A retrieval means for retrieving previous examination information at the position of the endoscope from the storage means based on the acquired identification information and the estimated position of the endoscope. A display control means for displaying the previous examination information on a display device in a case where the retrieved previous examination information includes the information regarding the lesion.

### TECHNICAL FIELD

The present disclosure relates to a technology for assisting endoscopy.

### BACKGROUND ART

At the time of endoscopy, a doctor may want to know the “past state of a patient's observed site”. For example, the doctor may want to observe the condition of previously detected polyps and the post-resection sites of polyps. In inflammatory bowel disease (IBD) (hereinafter referred to as “IBD”), sites with previous inflammation are considered to carry a high risk of developing colorectal cancer. Therefore, in a case where the patient has IBD, the doctor may want to know sites with previous inflammation during endoscopy. However, since it is necessary to refer to the electronic medical records in order to know the “past state of a patient's observed site”, it is difficult to know the “past state of a patient's observed site” during endoscopy. Patent Document 1 proposes an endoscopic imaging site identification device capable of displaying the past image of the current endoscopic observation site in such a way that the current image can be compared with the past image.

### PRECEDING TECHNICAL REFERENCE

### Patent Document

Patent Document 1: JP 2016-189812A

### SUMMARY

### Problem to be Solved

However, even with Patent Document 1, the doctor cannot appropriately display the state of the past observed site corresponding to the current observed site.

An object of the present disclosure is to provide an endoscopy support device capable of displaying information from the previous examination corresponding to a current observed site in endoscopy.

### Means for Solving the Problem

According to an example aspect of the present invention, there is provided an endoscopy support device, including:
an image acquisition means for acquiring an endoscopic image captured by an endoscope;
an identification information acquisition means for acquiring identification information of a patient;
a position estimation means for estimating a position of the endoscope in a large intestine;
a storage means for storing previous examination information associating the identification information of the patient, a position where a lesion was detected in a previous examination, an image of the lesion, and information regarding the lesion;
a retrieval means for retrieving previous examination information at the position of the endoscope from the storage means based on the acquired identification information and the estimated position of the endoscope; and
a display control means for displaying the previous examination information on a display device in a case where the retrieved previous examination information includes the information regarding the lesion.

According to another example aspect of the present invention, there is provided an endoscopy support method including:
acquiring an endoscopic image captured by an endoscope;
acquiring identification information of a patient;
estimating a position of the endoscope in a large intestine;
retrieving previous examination information related to the acquired identification information and the estimated position of the endoscope from a storage means for storing previous examination information associating the identification information of the patient, a position where a lesion was detected in a previous examination, an image of the lesion, and information regarding the lesion; and
displaying the previous examination information on a display device in a case where the retrieved previous examination information includes the information regarding the lesion.

According to a further example aspect of the present invention, there is provided a recording medium recording a program for causing a computer to execute processing including:
acquiring an endoscopic image captured by an endoscope;
acquiring identification information of a patient;
estimating a position of the endoscope in a large intestine;
retrieving previous examination information related to the acquired identification information and the estimated position of the endoscope from a storage means for storing previous examination information associating the identification information of the patient, a position where a lesion was detected in a previous examination, an image of the lesion, and information regarding the lesion; and
displaying the previous examination information on a display device in a case where the retrieved previous examination information includes the information regarding the lesion.

### Effect

According to the present disclosure, in endoscopy, it is possible to display information from the previous examination corresponding to a current observed site.

### BRIEF DESCRIPTION OF THE DRAWINGS

FIG. 1 is a block diagram illustrating a schematic configuration of an endoscopy system.

FIG. 2 is a block diagram illustrating a hardware configuration of an endoscopy support device.

FIG. 3 is a block diagram illustrating a functional configuration of an endoscopy support device according to a first example embodiment.

FIG. 4 schematically illustrates a structure of a large intestine.

FIG. 5 illustrates an example of a data structure of previous examination information according to the first example embodiment.

FIG. 6 illustrates a display example by a display device according to the first example embodiment.

FIG. 7 illustrates another display example by a display device according to the first example embodiment.

FIG. 8 is a flowchart illustrating image display processing by an endoscopy support device according to the first example embodiment.

FIG. 9 is a block diagram illustrating a functional configuration of an endoscopy support device according to a second example embodiment.

FIG. 10 illustrates another example of a data structure of previous examination information according to the second example embodiment.

FIG. 11 illustrates an example of a method for determining a progression state of inflammation.

FIG. 12 illustrates still another display example by a display device according to the second example embodiment.

FIG. 13 illustrates still another display example by a display device according to the second example embodiment.

FIG. 14 illustrates still another display example by a display device according to the second example embodiment.

FIG. 15 is a flowchart illustrating image display processing by an endoscopy support device according to the second example embodiment.

FIG. 16 is a block diagram illustrating a functional configuration of an endoscopy support device according to a third example embodiment.

FIG. 17 is a flowchart illustrating processing by an endoscopy support device according to the third example embodiment.

### EXAMPLE EMBODIMENTS

Hereinafter, preferred example embodiments of the present disclosure will be described with reference to the drawings.

### First Example Embodiment

### [System Configuration]

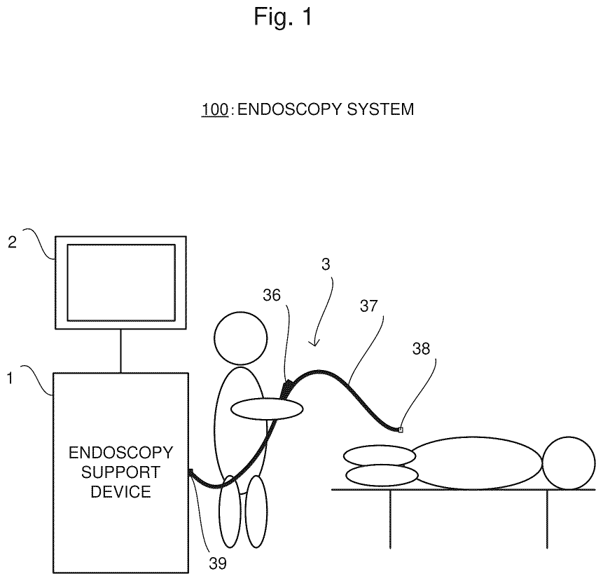

FIG. 1 illustrates a schematic configuration of an endoscopy system 100. The endoscopy system 100 estimates the position of the endoscope in the large intestine during endoscopic examination (including treatment). The endoscopy system 100 retrieves information regarding the previous examination at the position of the endoscope (hereinafter also referred to as “previous examination information”) from a database. At this time, in a case where lesion information regarding a lesion is included in the previous examination information, the endoscopy system 100 displays the previous examination information on the display device. The lesion information includes, for example, information such as “polyp present” or “polyp resected”. On the other hand, in a case where lesion information is not included in the previous examination information, the endoscopy system 100 does not display the previous examination information on the display device.

As described above, in a case where the endoscope reaches a location where the lesion was found in the previous examination or a location where the treatment was performed during the previous examination, the endoscopy system 100 displays the previous examination information on the display device. Thus, the doctor can easily monitor lesions and post-treatment progress.

As illustrated in FIG. 1, the endoscopy system 100 mainly includes an endoscopy support device 1, a display device 2, and an endoscopic scope 3 connected to the endoscopy support device 1.

The endoscopy support device 1 acquires a video (that is, a moving image.

Hereinafter also referred to as “endoscopic video Ic”) captured by the endoscopic scope 3 during the endoscopy from the endoscopic scope 3, and causes the display device 2 to display data for confirmation by an examiner (doctor) performing the endoscopy. Specifically, the endoscopy support device 1 acquires a moving image of an internal organ captured by the endoscopic scope 3 during the endoscopy as the endoscopic video Ic. In a case where the doctor finds a lesion during the endoscopy, the doctor operates the endoscopic scope 3 to input an instruction to image a lesion site. The endoscopy support device 1 generates a lesion image showing the lesion site based on an imaging instruction from the doctor. Specifically, the endoscopy support device 1 generates a lesion image, which is a still image, from an endoscopic video Ic, which is a moving image, based on the imaging instruction from the doctor.

The display device 2 is a display or the like that performs a predetermined display based on a display signal supplied from the endoscopy support device 1.

The endoscopic scope 3 mainly includes an operation unit 36 for the doctor to perform input such as air supply, water supply, angle adjustment, and imaging instructions, a shaft 37 that has flexibility and is inserted into an organ of a subject to be examined, a distal end portion 38 incorporating an imaging unit such as an ultra-small imaging element, and a connection unit 39 for connecting to the endoscopy support device 1.

In the following description, the description will mainly focus on the processing during the large intestine endoscopy, but the examination target is not limited to the large intestine, and may be a digestive tract (gastrointestinal organ) such as the stomach, esophagus, small intestine, or duodenum.

### [Hardware Configuration]

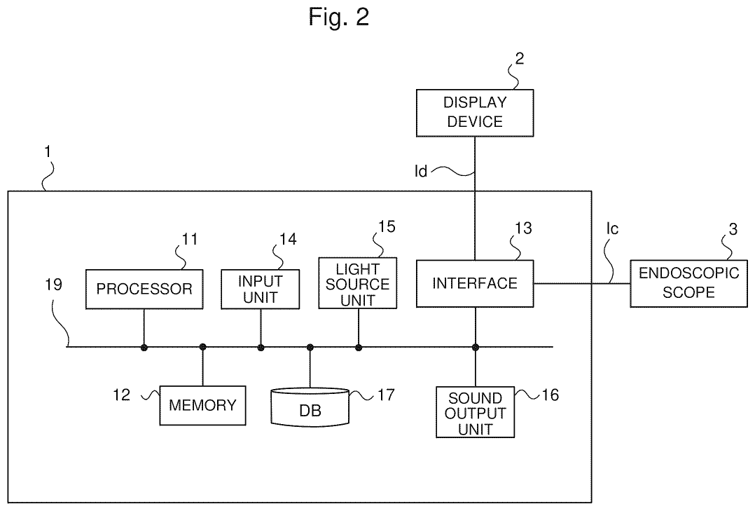

FIG. 2 illustrates a hardware configuration of the endoscopy support device 1. The endoscopy support device 1 mainly includes a processor 11, a memory 12, an interface 13, an input unit 14, a light source unit 15, a sound output unit 16, and a database (hereinafter referred to as “DB”) 17. These elements are connected via a data bus 19.

The processor 11 executes a predetermined processing by executing a program or the like stored in the memory 12. The processor 11 is, for example, a processor such as a central processing unit (CPU), a graphics processing unit (GPU), or a tensor processing unit (TPU). The processor 11 may include a plurality of processors. The processor 11 is an example of a computer.

The memory 12 includes various volatile memories used as a working memory, such as a random access memory (RAM) and a read only memory (ROM), and a nonvolatile memory that stores information necessary for processing of the endoscopy support device 1. The memory 12 may include an external storage device such as a hard disk connected to or incorporated in the endoscopy support device 1, or may include a storage medium such as a removable flash memory or a disk medium. The memory 12 stores a program for the endoscopy support device 1 to execute each process in the present example embodiment.

The memory 12 temporarily stores a series of endoscopic videos Ic captured by the endoscopic scope 3 in the endoscopy under the control of the processor 11. The memory 12 temporarily stores a lesion image captured based on an imaging instruction of a doctor during endoscopy. These images are stored in the memory 12 in association with, for example, identification information of the subject (for example, a patient ID), information regarding a time stamp, and the like.

The interface 13 performs an interface operation between the endoscopy support device 1 and an external device. For example, the interface 13 supplies display data Id generated by the processor 11 to the display device 2. The interface 13 supplies illumination light generated by the light source unit 15 to the endoscopic scope 3. The interface 13 supplies an electrical signal indicating the endoscopic video Ic supplied from the endoscopic scope 3 to the processor 11. The interface 13 may be a communication interface such as a network adapter for performing wired or wireless communication with the external device, or may be a hardware interface conforming to a universal serial bus (USB), serial AT attachment (SATA), or the like.

The input unit 14 generates an input signal based on the operation by the doctor. The input unit 14 is, for example, a button, a touch panel, a remote controller, a voice input device, or the like. The light source unit 15 generates light to be supplied to the distal end portion 38 of the endoscopic scope 3. The light source unit 15 may also incorporate a pump or the like for delivering water or air to be supplied to the endoscopic scope 3. The sound output unit 16 outputs sound under the control of the processor 11.

The DB 17 stores the previous examination information of the subject. The previous examination information includes a location where a lesion is detected, a lesion image, and lesion information. The DB 17 may include an external storage device such as a hard disk connected to or incorporated in the endoscopy support device 1, or may include a storage medium such as a removable flash memory. Instead of providing the DB 17 in the endoscopy system 100, the DB 17 may be provided on an external server or the like, and the previous examination information may be acquired from the server by communication.

### [Functional Configuration]

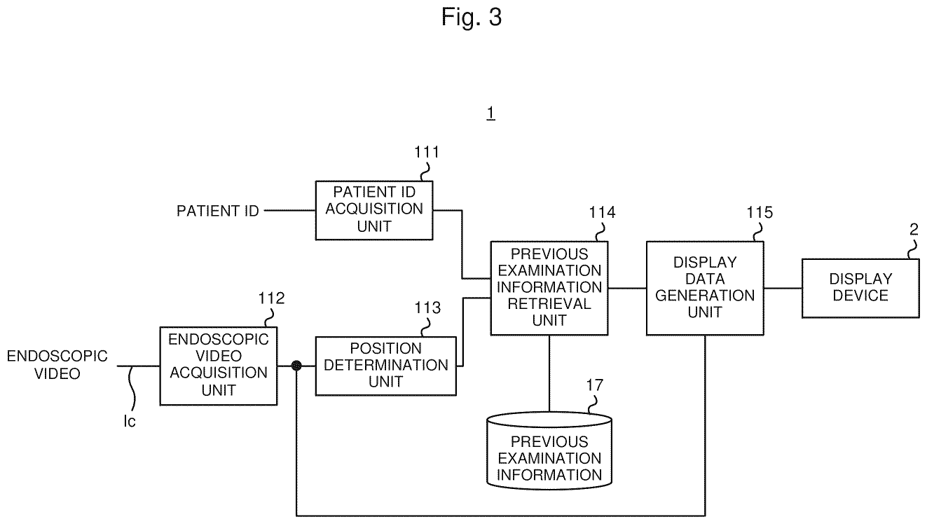

FIG. 3 is a block diagram illustrating a functional configuration of the endoscopy support device 1 according to the first example embodiment. The endoscopy support device 1 functionally includes a patient ID acquisition unit 111, an endoscopic video acquisition unit 112, a position determination unit 113, a previous examination information retrieval unit 114, and a display data generation unit 115 in addition to the display device 2 and the DB 17 described above.

The patient ID of the patient who is the subject is input to the endoscopy support device 1 through the input unit 14. The patient ID is information for uniquely identifying a patient, and an individual identification number such as My Number (Japan's Social Security and Tax Number) may be used in addition to an ID uniquely assigned to each patient. The patient ID acquisition unit 111 acquires the patient ID input through the input unit 14, and outputs the patient ID to the previous examination information retrieval unit 114.

The endoscopic video Ic is input from the endoscopic scope 3 to the endoscopy support device 1. The endoscopic video Ic is input to the endoscopic video acquisition unit 112. The endoscopic video acquisition unit 112 outputs the input endoscopic video Ic to the position determination unit 113 and the display data generation unit 115.

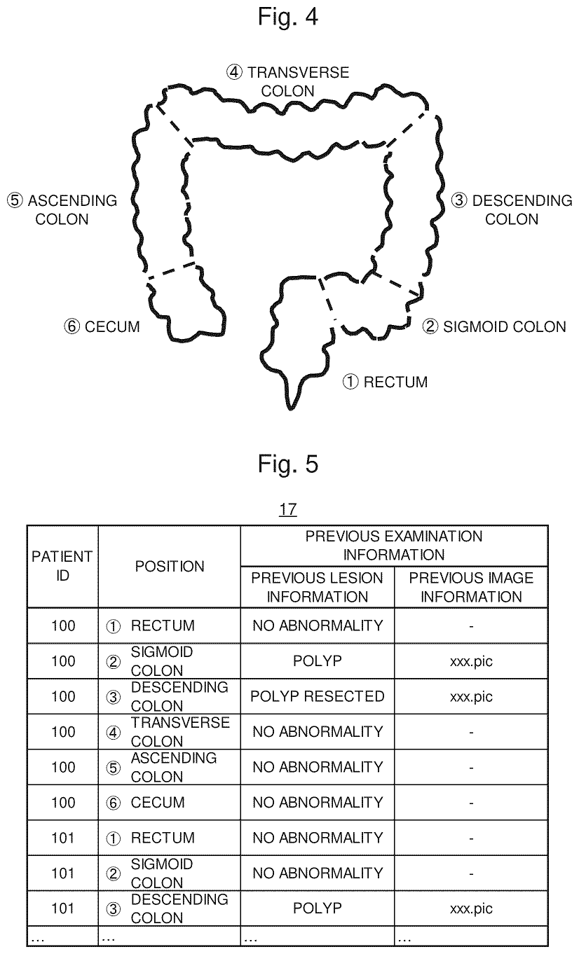

The position determination unit 113 determines the position of the endoscopic scope 3, that is, the imaging position of the endoscopic video, based on the endoscopic video Ic input from the endoscopic video acquisition unit 112. For example, as illustrated in FIG. 4, the large intestine includes a plurality of sections such as a rectum, a sigmoid colon, a descending colon, a transverse colon, an ascending colon, and a cecum. The position determination unit 113 determines which section the imaging position belongs to.

Specifically, at the boundary of each section of the large intestine, there is a portion (hereinafter also referred to as “landmark”) where the endoscopic scope 3 comes into contact with the intestinal wall and bends. The position determination unit 113 can determine which section of the large intestine the imaging position belongs to based on the landmark. For example, the position determination unit 113 can determine the imaging position using the DB in which the image of the landmark and the section of the large intestine are registered in association with each other. The position determination unit 113 extracts a still image (hereinafter also referred to as “endoscopic image”) from the endoscopic video, and calculates similarity between the endoscopic image and the image of the landmark registered in the DB. In a case where the image of the landmark whose similarity to the endoscopic image is equal to or more than a predetermined threshold is detected, the position determination unit 113 acquires a section of the large intestine corresponding to the image of the landmark from the DB. The position determination unit 113 outputs the section of the large intestine acquired from the DB as the imaging position to the previous examination information retrieval unit 114. In a case where the image of the landmark whose similarity to the endoscopic image is equal to or more than a predetermined threshold is not detected, the position determination unit 113 outputs a result indicating that there is no change in the imaging position to the previous examination information retrieval unit 114.

The position determination unit 113 may determine the imaging position of the endoscopic video using an image recognition model prepared in advance or the like. This image recognition model is a model trained in advance to detect the landmark from the endoscopic video and determine the imaging position.

The previous examination information retrieval unit 114 retrieves the previous examination information from the DB 17 based on the patient ID input from the patient ID acquisition unit 111 and the imaging position input from the position determination unit 113.

FIG. 5 illustrates an example of a data structure of the previous examination information stored in the DB 17. As illustrated in FIG. 5, the position and the previous examination information are stored in the DB 17 in association with the patient ID. The “position” is information indicating each section (any of rectum, sigmoid colon, descending colon, transverse colon, ascending colon, and cecum) of the large intestine. The “previous examination information” includes previous lesion information and previous image information. The “previous lesion information” indicates a found lesion or a treatment performed on the lesion. “No abnormality” indicates that there is no lesion at the corresponding section. The “previous image information” is an endoscopic image obtained by imaging a lesion, a site on which treatment is performed (hereinafter also referred to as “treatment site”), or the like. In FIG. 5, the previous image information is indicated by a file name of image data. “-(hyphen)” indicates that there is no lesion or treatment site at the corresponding site and there is no endoscopic image at the corresponding site. In the example of FIG. 5, one piece of previous lesion information is illustrated for one section of the large intestine. However, in a case where there are a plurality of lesions or a plurality of treatment sites for one section of the large intestine, a plurality of pieces of previous lesion information and a plurality of pieces of previous image information are stored in the DB 17.

Returning to FIG. 3, the previous examination information retrieval unit 114 acquires the previous examination information corresponding to a combination of the patient ID and the imaging position from the DB 17. The previous examination information retrieval unit 114 outputs the imaging position and the previous examination information to the display data generation unit 115.

The display data generation unit 115 generates display data Id by using the endoscopic video Ic input from the endoscopic video acquisition unit 112, the imaging position input from the previous examination information retrieval unit 114, and the previous examination information, and outputs the display data Id to the display device 2. In a case where information regarding a lesion and treatment is not included in the previous examination information (that is, the previous lesion information is “no abnormality”), the display data generation unit 115 generates display data Id by using the endoscopic video Ic and the imaging position, and outputs the display data Id to the display device 2.

In the above-described configuration, the patient ID acquisition unit 111 is an example of an identification information acquisition means, the endoscopic video acquisition unit 112 is an example of an image acquisition means, the position determination unit 113 is an example of a position estimation means, the previous examination information retrieval unit 114 is an example of a retrieval means, and the display data generation unit 115 is an example of a display control means.

### [Display Example]

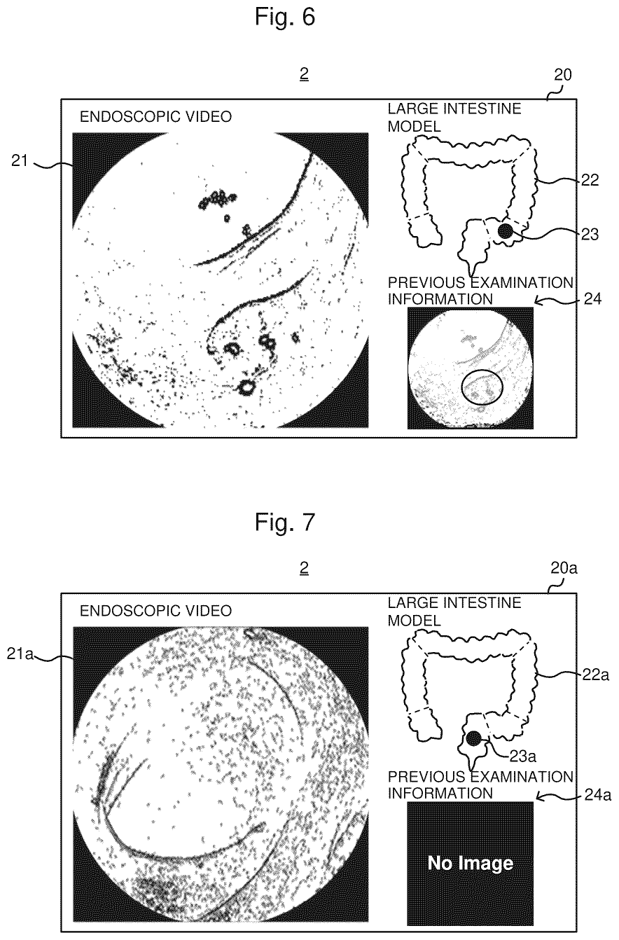

Next, a display example by the display device 2 will be described. FIG. 6 illustrates an example of the display example by the display device 2. In the display example of FIG. 6, an endoscopic video 21, a large intestine model 22, an imaging position 23, and a previous examination information area 24 are displayed in a display area 20.

The endoscopic video 21 is an endoscopic video Ic during examination. The large intestine model 22 shows a schematic view of the large intestine. On the large intestine model 22, the current imaging position 23 is indicated by a black circle. In FIG. 6, the imaging position 23 is illustrated at a section corresponding to the sigmoid colon of the large intestine model 22. The previous examination information area 24 is an area for displaying the previous examination information at the imaging position 23. In FIG. 6, an endoscopic image including a lesion is displayed in the previous examination information area 24 as previous examination information for the sigmoid colon. With the display as illustrated in FIG. 6, the doctor can recognize that the current imaging position is in the sigmoid colon and the lesion was detected in the sigmoid colon in the previous examination.

In FIG. 6, one image is displayed in the previous examination information area 24, but in a case where there are two or more lesions or treatment sites for one section, two or more images may be displayed depending on the available space of the previous examination information area 24. A display space of the previous examination information area 24 may be secured by adding a display device to display the endoscopic video 21 on one screen and display the large intestine model 22 and the previous examination information area 24 on the other screen.

FIG. 7 illustrates another display example by the display device 2. In the display example of FIG. 7, an endoscopic video 21a, a large intestine model 22a, an imaging position 23a, and a previous examination information area 24a are displayed in a display area 20a. In FIG. 7, the examination proceeds from the state of FIG. 6, and the imaging position is moved from the sigmoid colon to the rectum. The previous examination information is not displayed in the previous examination information area 24a, indicating that there was no lesion, treatment site, or the like in the rectum in the previous examination.

As described above, by displaying the previous examination information at the current imaging position, the doctor can perform the endoscopy while performing comparison with the previous examination information.

### [Image Display Processing]

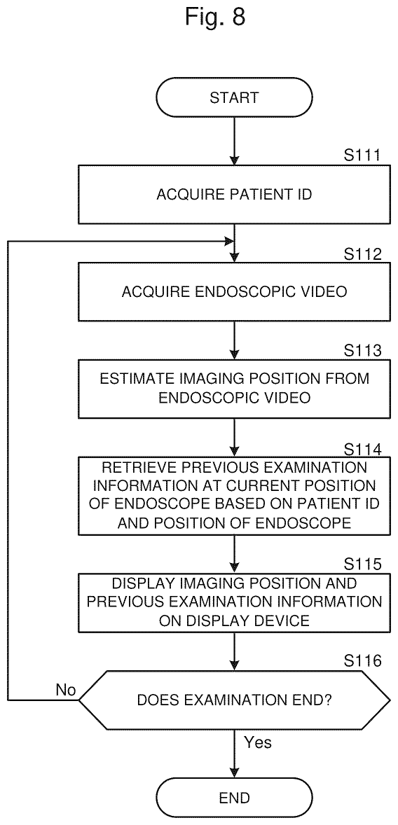

Next, image display processing as described above will be described. FIG. 8 is a flowchart of image display processing by the endoscopy support device 1. This processing is implemented by the processor 11 illustrated in FIG. 2 executing a program prepared in advance and operating as each element illustrated in FIG. 3.

First, the patient ID of the patient who is the subject is input to the endoscopy support device 1 through the input unit 14. The patient ID acquisition unit 111 acquires the patient ID, and outputs the patient ID to the previous examination information retrieval unit 114 (step S111). Next, the endoscopic video Ic is input from the endoscopic scope 3 to the endoscopy support device 1. The endoscopic video acquisition unit 112 acquires endoscopic video Ic and outputs the acquired endoscopic video Ic to the position determination unit 113 and the display data generation unit 115 (step S112).

Next, based on the endoscopic video Ic input from the endoscopic video acquisition unit 112, the position determination unit 113 determines which site (rectum, sigmoid colon, descending colon, transverse colon, ascending colon, or cecum) of the large intestine the imaging position of the endoscopic video belongs to. The position determination unit 113 outputs the determined imaging position to the previous examination information retrieval unit 114 (step S113).

Next, the previous examination information retrieval unit 114 acquires, from the DB 17, the previous examination information corresponding to a combination of the patient ID input from the patient ID acquisition unit 111 and the imaging position input from the position determination unit 113. The previous examination information retrieval unit 114 outputs the imaging position and the previous examination information to the display data generation unit 115 (step S114).

The display data generation unit 115 generates display data Id by using the endoscopic video Ic, the imaging position, and the previous examination information, and outputs the display data Id to the display device 2 (step S115). In a case where information regarding a lesion and treatment is not included in the previous examination information, the display data generation unit 115 generates display data Id by using the endoscopic video Ic and the imaging position, and outputs the display data Id to the display device 2.

Next, the endoscopy support device 1 determines whether the examination ends. The endoscopy support device 1 determines that the examination ends, for example, in a case where the doctor performs an operation for ending the endoscopy support device 1 or the endoscopic scope 3 in the examination. The endoscopy support device 1 may determine that the examination automatically ends in a case where a captured image becomes an image of an area outside the organ through image analysis of an image captured using the endoscopic scope 3. In a case where it is determined that the examination is not ended (step S116: No), the processing returns to step S112. On the other hand, in a case where it is determined that the examination ends (step S116: Yes), the processing ends.

### MODIFICATION EXAMPLE

Next, a modification example of the first example embodiment will be described. The following modification example can be appropriately combined and applied to the first example embodiment.

### Modification Example

In the first example embodiment, the position determination unit 113 determines the imaging position based on the endoscopic video, but the position determination unit 113 may determine the imaging position using a magnetic coil. By incorporating the magnetic coil in the distal end portion 38 of the endoscopic scope 3, the position determination unit 113 can estimate a three-dimensional insertion shape of the endoscopic scope 3 using magnetism generated from the magnetic coil. The position determination unit 113 may determine which section of the large intestine the imaging position belongs to based on the estimated insertion shape. The position determination unit 113 may determine which section of the large intestine the imaging position belongs to by using the insertion length of the endoscopic scope 3 inserted into the large intestine in addition to the insertion shape of the endoscopic scope 3.

### Second Example Embodiment

Next, a second example embodiment will be described. An endoscopy system according to a second example embodiment is different from the endoscopy system 100 according to the first example embodiment in that the examination can proceed while comparing the inflammatory state of the large intestine in the current examination with the inflammatory state of the large intestine in the previous examination. A system configuration and a hardware configuration of the endoscopy system according to the second example embodiment are similar to those of the endoscopy system 100 according to the first example embodiment, and thus the description thereof will be omitted.

### [Functional Configuration]

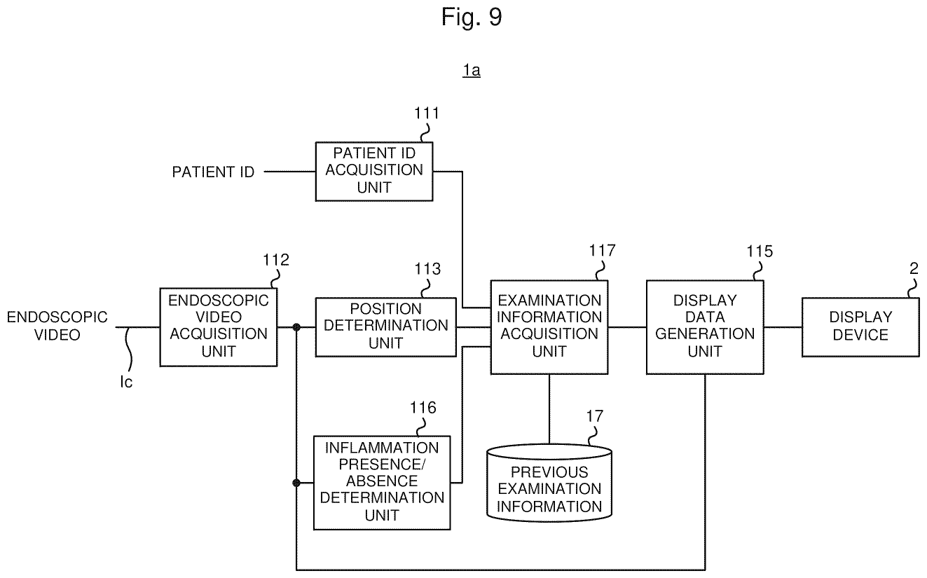

FIG. 9 is a block diagram illustrating a functional configuration of an endoscopy support device la according to the second example embodiment. The endoscopy support device la functionally includes a patient ID acquisition unit 111, an endoscopic video acquisition unit 112, a position determination unit 113, a display data generation unit 115, an inflammation presence/absence determination unit 116, and an examination information acquisition unit 117 in addition to the display device 2 and the DB 17 described above.

The patient ID acquisition unit 111, the endoscopic video acquisition unit 112, and the position determination unit 113 have configurations similar to those of the patient ID acquisition unit 111, the endoscopic video acquisition unit 112, and the position determination unit 113 of the endoscopy support device 1 according to the first example embodiment, and operate similarly. Therefore, the description thereof will be omitted.

The endoscopic video Ic is input from the endoscopic video acquisition unit 112 to the inflammation presence/absence determination unit 116. The inflammation presence/absence determination unit 116 determines the presence or absence of inflammation from the endoscopic video Ic using an image recognition model prepared in advance or the like. This image recognition model is a model trained in advance to estimate the presence or absence of inflammation using an endoscopic image as an input, and is hereinafter also referred to as an inflammation estimation model.

Specifically, the inflammation presence/absence determination unit 116 extracts an endoscopic image from the endoscopic video and inputs the endoscopic image to the inflammation estimation model. The inflammation estimation model estimates the presence or absence of inflammation from the endoscopic image and outputs a confidence score. The confidence score is, for example, equal to or more than zero and equal to or less than one, and the closer the confidence score is to one, the higher the possibility of inflammation. The inflammation presence/absence determination unit 116 determines that there is inflammation in a case where the confidence score is equal to or more than a predetermined threshold. On the other hand, the inflammation presence/absence determination unit 116 determines that there is no inflammation in a case where the confidence score is less than the predetermined threshold. The inflammation presence/absence determination unit 116 outputs the result of the inflammation presence/absence determination to the examination information acquisition unit 117. As the inflammation estimation model, a model trained in advance using training data generated by labeling the presence or absence of inflammation for a large number of images showing an inflammatory state and a large number of images showing a non-inflammatory state can be used.

The examination information acquisition unit 117 receives the patient ID from the patient ID acquisition unit 111, the imaging position from the position determination unit 113, and the result of the inflammation presence/absence determination from the inflammation presence/absence determination unit 116.

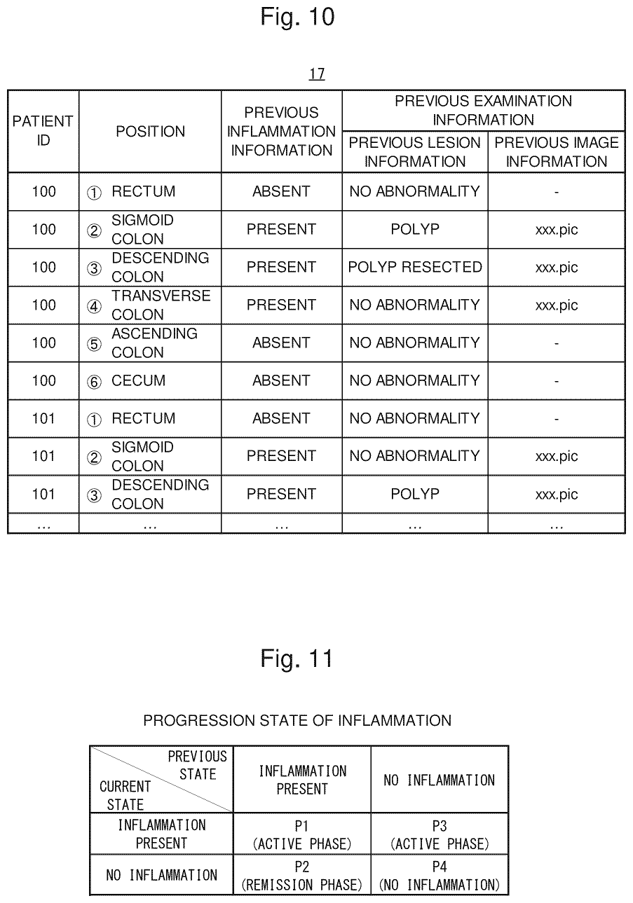

FIG. 10 illustrates an example of a data structure of the previous examination information stored in the DB 17. As illustrated in FIG. 10, a position, a previous inflammation information, and a previous examination information are stored in the DB 17 in association with the patient ID. Since the “position” is similar to the “position” of the previous examination information illustrated in FIG. 5, the description thereof will be omitted. The “previous inflammation information” is information indicating the presence or absence of inflammation in the previous examination. In FIG. 10, “present” is shown in a case where there was inflammation in the previous examination, and “absent” is shown in a case where there was no inflammation in the previous examination. The “previous examination information” includes previous lesion information and previous image information. Since the “previous lesion information” is similar to the “previous lesion information” of the previous examination information illustrated in FIG. 5, the description thereof will be omitted. The “previous image information” is an endoscopic image obtained by imaging a lesion, a treatment site, inflammation or the like. In FIG. 10, the previous image information is indicated by a file name of image data. “-(hyphen)” indicates that there is no lesion, treatment site, or inflammation at the corresponding site and there is no endoscopic image at the corresponding site. In the example of FIG. 10, one piece of previous lesion information is illustrated for one section of the large intestine. However, in a case where there are a plurality of lesions or a plurality of treatment sites for one section of the large intestine, a plurality of pieces of previous lesion information and a plurality of pieces of previous image information are stored in the DB 17.

Returning to FIG. 9, the examination information acquisition unit 117 acquires the previous inflammation information corresponding to the patient ID from the DB 17. The examination information acquisition unit 117 determines the progression state of inflammation at the imaging position based on the imaging position, the result of the inflammation presence/absence determination, and the previous inflammation information.

FIG. 11 illustrates an example of a method for determining the progression state of inflammation. In FIG. 11, the progression state of inflammation is classified into “P1 (active phase)”, “P2 (remission phase)”, “P3 (active phase)”, and “P4 (no inflammation)”. In a case where inflammation is present in a site of a large intestine in the current examination and the previous examination, the examination information acquisition unit 117 determines the progression state of inflammation as “P1 (active phase)”. In a case where inflammation is not present in a site of a large intestine in the current examination, but it was present in the previous examination, the examination information acquisition unit 117 determines the progression state of inflammation as “P2 (remission phase)”. In a case where inflammation is present in a site of a large intestine in the current examination, but it was not present in the previous examination, the examination information acquisition unit 117 determines the progression state of inflammation as “P3 (active phase)”. In a case where inflammation is not present in a site of a large intestine in the current examination and the previous examination, the examination information acquisition unit 117 determines the progression state of inflammation as “P4 (no inflammation)”.

The examination information acquisition unit 117 acquires the previous examination information corresponding to a combination of the patient ID and the imaging position from the DB 17. The examination information acquisition unit 117 outputs the imaging position, the result of the inflammation presence/absence determination, the previous inflammation information, the previous examination information, and the progression state of inflammation to the display data generation unit 115.

The display data generation unit 115 generates the display data Id by using the endoscopic video Ic input from the endoscopic video acquisition unit 112, the imaging position input from the examination information acquisition unit 117, the result of the inflammation presence/absence determination, the previous inflammation information, the previous examination information, and the progression state of inflammation, and outputs the display data Id to the display device 2. In a case where information regarding a lesion, treatment, and inflammation is not included in the previous examination information (that is, the previous lesion information is “no abnormality” and the previous inflammation information is “absent”), the display data generation unit 115 generates the display data Id by using the endoscopic video Ic, the imaging position, the result of the inflammation presence/absence determination, the previous inflammation information, and the progression state of inflammation, and outputs the display data Id to the display device 2.

### Display Example

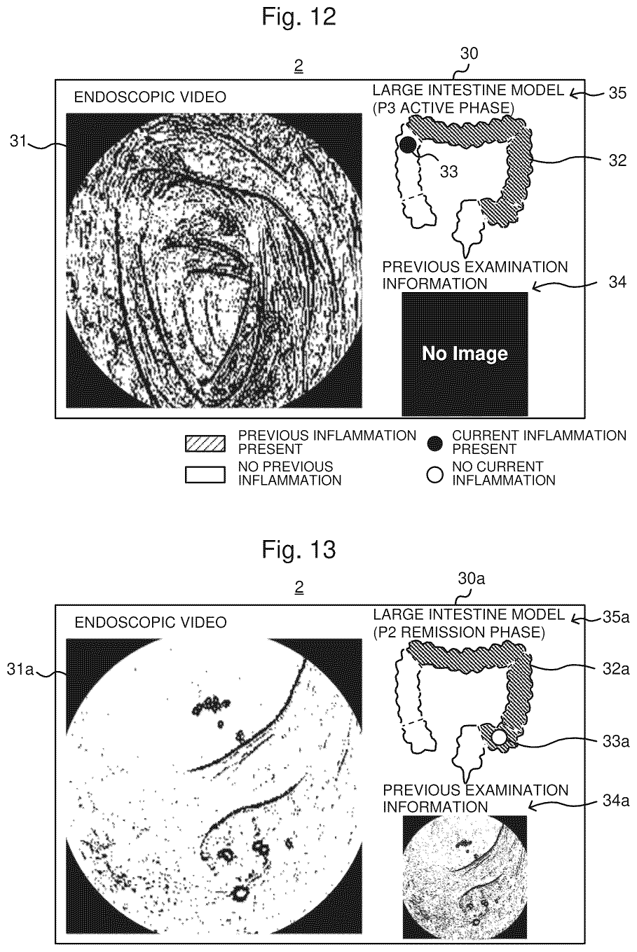

Next, a display example by the display device 2 will be described. FIG. 12 illustrates an example of the display example by the display device 2. In the display example of FIG. 12, an endoscopic video 31, a large intestine model 32, an imaging position 33, and a previous examination information area 34, and an inflammation progression state 35 are displayed in a display area 30.

The endoscopic video 31 is an endoscopic video Ic during examination. The large intestine model 32 shows a schematic view of the large intestine. The display mode of each site (rectum, sigmoid colon, descending colon, transverse colon, ascending colon, cecum) in the schematic view of the large intestine differs depending on whether inflammation is present at the time of the previous examination. In FIG. 12, a site with inflammation in the previous examination is indicated by oblique hatching, and a site without inflammation in the previous examination is indicated in white. With the large intestine model 32 in FIG. 12, the doctor can recognize that inflammation was present in the sigmoid colon, the descending colon, and the transverse colon in the previous examination.

On the large intestine model 32, the current imaging position 33 is indicated. The display mode of the imaging position 33 differs depending on whether inflammation is present at the current imaging position. For example, in a case where inflammation is present at the current imaging position, the imaging position 33 is indicated by a black circle. In a case where inflammation is not present at the current imaging position, the imaging position 33 is indicated by a white circle. With the large intestine model and the imaging position 33 in FIG. 12, the doctor can recognize that the current imaging position is the ascending colon and inflammation is present in the current ascending colon.

The previous examination information area 34 is an area for displaying the previous examination information at the current imaging position 33. In FIG. 12, the previous examination information is not displayed in the previous examination information area 34 and indicates that there was no lesion, treatment site, or inflammation in the ascending colon in the previous examination.

The inflammation progression state 35 indicates the progression state of inflammation at the imaging position 33. FIG. 12 illustrates that the progression state of inflammation in the ascending colon is “P3 (active phase)”. With such a display, the doctor can recognize that the range of inflammation is expanded.

FIG. 13 illustrates still another display example by the display device 2. In the display example of FIG. 13, an endoscopic video 31a, a large intestine model 32a, an imaging position 33a, a previous examination information area 34a, and an inflammation progression state 35a are displayed in a display area 30a. In FIG. 13, the examination proceeds from the state of FIG. 12, and the imaging position is moved from the ascending colon to the sigmoid colon.

In FIG. 13, the imaging position 33a is a white circle, and indicates that inflammation is not present in the current sigmoid colon. In the previous examination information area 34a, an endoscopic image of the sigmoid colon in the previous examination is displayed. The inflammation progression state 35a indicates that the progression state of inflammation in the sigmoid colon is “P2 (remission phase)”. With such a display, the doctor can recognize that the sigmoid colon is a site with inflammation at the time of the previous examination, and it is necessary to carefully observe the site.

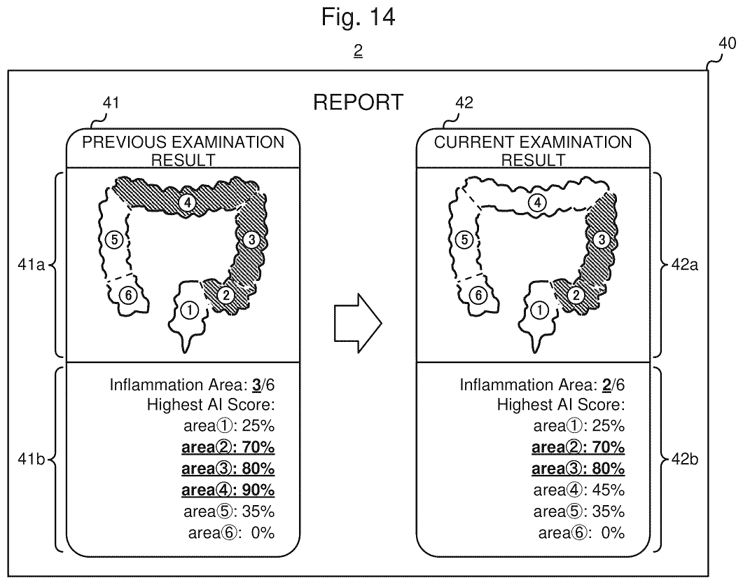

FIG. 14 illustrates still another display example by the display device 2. FIG. 14 is a report for comparing the current examination result with the previous examination result, and the report is output after the examination using the endoscope ends. In the display example of FIG. 14, a previous examination result 41 and a current examination result 42 are displayed in a display area 40.

The previous examination result 41 indicates a previous examination result. The previous examination result 41 includes a large intestine model 41a and detailed information 41b.

The large intestine model 41a shows a schematic view of the large intestine. In FIG. 14, numerals from one to six are displayed at respective sections of the large intestine model 41a, one indicates a rectum, two indicates a sigmoid colon, three indicates a descending colon, four indicates a transverse colon, five indicates an ascending colon, and six indicates a cecum. For each section of the large intestine model 41a, a site with inflammation in the previous examination is indicated by oblique hatching, and a site without inflammation in the previous examination is indicated in white. In the large intestine model 41a of FIG. 14, the sigmoid colon of numeral 2, the descending colon of numeral 3, and the transverse colon of numeral 4 are indicated by oblique hatching.

The detailed information 41b indicates details of the previous examination result. In the detailed information 41b, the ratio of the sites with inflammation and the confidence score of each site are displayed. In the detailed information 41b of FIG. 14, the confidence score is represented by a value ranging from 0% to 100%, and a site where the confidence score is equal to or more than a predetermined threshold is indicated as an inflamed site with bold text and an underline. It can be recognized from the detailed information 41b of FIG. 14 that the ratio of the sites with inflammation in the previous examination is three out of six sites, and the sites with inflammation in the previous examination are the sigmoid colon, the descending colon, and the transverse colon.

The current examination result 42 indicates a current examination result. The current examination result 42 includes a large intestine model 42a and detailed information 42b.

In the current examination result 42, for each section of the large intestine model 42a, a site with inflammation in the current examination is indicated by oblique hatching, and a site without inflammation in the current examination is indicated in white. In the large intestine model 42a of FIG. 14, the sigmoid colon of numeral 2 and the descending colon of numeral 3 are indicated by oblique hatching.

The detailed information 42b indicates details of the current examination result. In the detailed information 42b, the ratio of the sites with inflammation and the confidence score of each site are displayed. It can be recognized from the detailed information 42b of FIG. 14 that the ratio of the sites with inflammation in the current examination is two out of six sites, and the sites with inflammation in the current examination are the sigmoid colon and the descending colon.

With the display as illustrated in FIG. 14, the doctor can easily compare the previous examination result with the current examination result.

### [Image Display Processing]

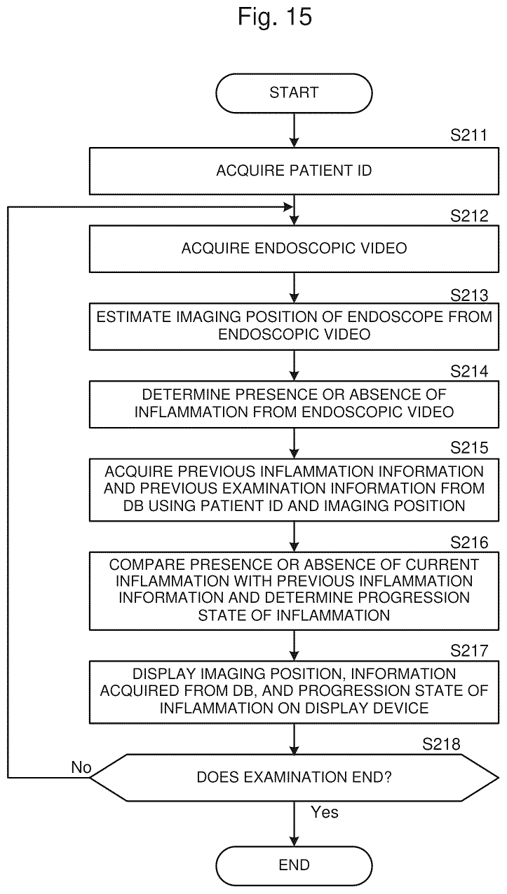

FIG. 15 is a flowchart of image display processing by the endoscopy support device la. This processing is implemented by the processor 11 illustrated in FIG. 2 executing a program prepared in advance and operating as each element illustrated in FIG. 9. The processing in steps S211 to S213 and step S218 is similar to the processing in steps S111 to S113 and step S117 of the first example embodiment illustrated in FIG. 8, and thus the description thereof will be omitted.

The endoscopic video Ic is input from the endoscopic video acquisition unit 112 to the inflammation presence/absence determination unit 116. The inflammation presence/absence determination unit 116 determines the presence or absence of inflammation from the endoscopic video Ic using the inflammation estimation model. The inflammation presence/absence determination unit 116 outputs the result of the inflammation presence/absence determination to the examination information acquisition unit 117 (step S214).

The examination information acquisition unit 117 receives the patient ID from the patient ID acquisition unit 111, the imaging position from the position determination unit 113, and the result of the inflammation presence/absence determination from the inflammation presence/absence determination unit 116. The examination information acquisition unit 117 acquires the previous inflammation information corresponding to the patient ID from the DB 17. The examination information acquisition unit 117 acquires previous lesion information and previous image information corresponding to a combination of the patient ID and the imaging position from the DB 17 (step S215).

Next, the examination information acquisition unit 117 determines the progression state of inflammation. Specifically, the examination information acquisition unit 117 determines the progression state of inflammation based on the imaging position, the result of the inflammation presence/absence determination, and the previous inflammation information. The examination information acquisition unit 117 outputs the imaging position, the result of the inflammation presence/absence determination, the previous inflammation information, the previous lesion information, the previous image information, and the progression state of inflammation to the display data generation unit 115 (step S216).

Next, the display data generation unit 115 generates the display data Id by using the endoscopic video Ic input from the endoscopic video acquisition unit 112, the imaging position input from the examination information acquisition unit 117, the result of the inflammation presence/absence determination, the previous inflammation information, the previous examination information, and the progression state of inflammation, and outputs the display data Id to the display device 2 (step S217). In a case where information regarding a lesion, treatment, and inflammation is not included in the previous examination information, the display data generation unit 115 generates the display data Id by using the endoscopic video Ic, the imaging position, the result of the inflammation presence/absence determination, the previous inflammation information, and the progression state of inflammation, and outputs the display data Id to the display device 2.

### Third Example Embodiment

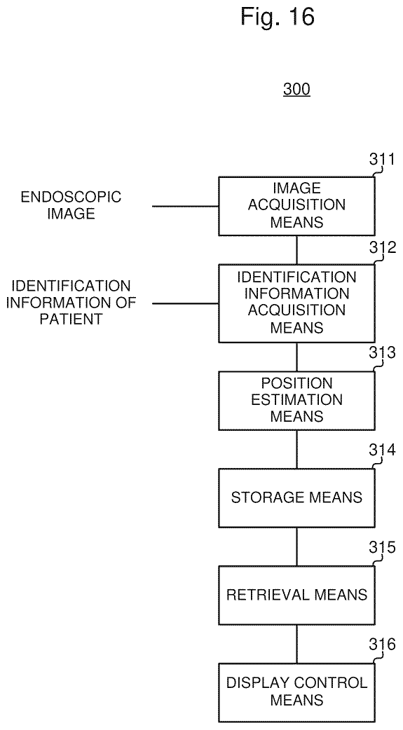

FIG. 16 is a block diagram illustrating a functional configuration of an endoscopy support device according to a third example embodiment. An endoscopy support device 300 includes an image acquisition means 311, an identification information acquisition means 312, a position estimation means 313, a storage means 314, a retrieval means 315, and a display control means 316.

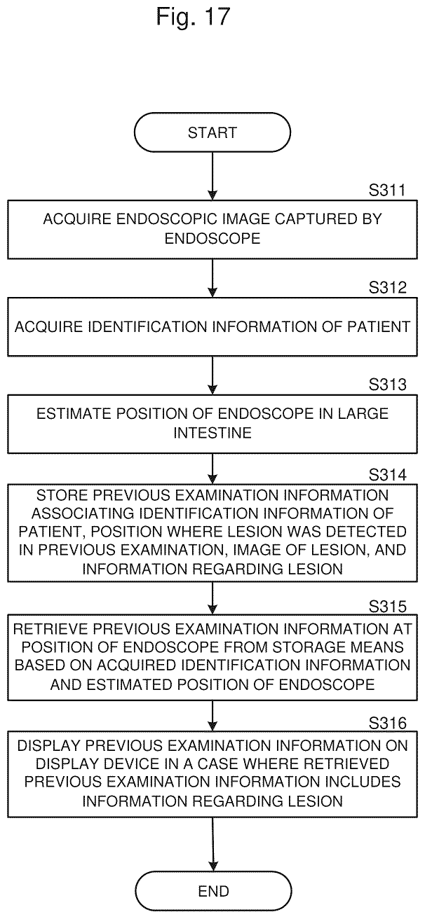

FIG. 17 is a flowchart of processing by the endoscopy support device according to the third example embodiment. The image acquisition means 311 acquires an endoscopic image captured by the endoscope (step S311). Next, the identification information acquisition means 312 acquires identification information of a patient (step S312). The position estimation means 313 estimates the position of the endoscope in the large intestine (step S313). The storage means 314 stores the previous examination information associating the identification information of a patient, a position where a lesion was detected in the previous examination, an image of the lesion, and information regarding the lesion (step S314). The retrieval means 315 retrieves the previous examination information at the position of the endoscope from the storage means based on the acquired identification information and the estimated position of the endoscope (step S315). In a case where the retrieved previous examination information includes information regarding a lesion, the display control means 316 displays the previous examination information on the display device (step S316).

In the endoscopy support device 300 according to the third example embodiment, in endoscopy, it is possible to display information from the previous examination corresponding to a current observed site.

Some or all of the above-described example embodiments may also be described as the following supplementary notes, but are not limited to the following supplementary notes.

### (Supplementary Note 1)

An endoscopy support device comprising:
an image acquisition means for acquiring an endoscopic image captured by an endoscope;
an identification information acquisition means for acquiring identification information of a patient;
a position estimation means for estimating a position of the endoscope in a large intestine;
a storage means for storing previous examination information associating the identification information of the patient, a position where a lesion was detected in a previous examination, an image of the lesion, and information regarding the lesion;
a retrieval means for retrieving previous examination information at the position of the endoscope from the storage means based on the acquired identification information and the estimated position of the endoscope; and
a display control means for displaying the previous examination information on a display device in a case where the retrieved previous examination information includes the information regarding the lesion.

### (Supplementary Note 2)

The endoscopy support device according to supplementary note 1, further comprising an inflammation presence or absence determination means for determining whether the large intestine is in an inflammatory state,
wherein the display control means displays information indicating the position of the endoscope and presence or absence of inflammation at the position of the endoscope on a model image showing an entire large intestine.

### (Supplementary Note 3)

The endoscopy support device according to supplementary note 2, wherein
the previous examination information includes information associating the identification information of the patient, a position where inflammation was detected in the previous examination, and an image of the inflammation, and
the display control means displays the previous examination information on the display device in a case where the retrieved previous examination information includes the image of the inflammation.

### (Supplementary Note 4)

The endoscopy support device according to supplementary note 3, wherein
the retrieval means retrieves the previous examination information based on the identification information of the patient,
the display control means generates a model image showing an entire large intestine in such a way as to be capable of identifying a position where previous inflammation was detected and a position where the previous inflammation was not detected, based on the previous examination information, and
the display control means displays information indicating a current position of the endoscope and presence or absence of inflammation at the position of the endoscope on the model image indicating the entire large intestine.

### (Supplementary Note 5)

The endoscopy support device according to supplementary note 4, wherein
the retrieval means determines a progression state of the inflammation based on a result of the inflammation presence or absence determination and the previous examination information, and
the display control means displays the progression state of the inflammation on the display device.

### (Supplementary Note 6)

The endoscopy support device according to supplementary note 1, wherein the position estimation means estimates the position of the endoscope in the large intestine from the endoscopic image.

### (Supplementary Note 7)

The endoscopy support device according to supplementary note 1, wherein the position estimation means estimates the position of the endoscope in the large intestine from a magnetic coil incorporated in the endoscope.

### (Supplementary Note 8)

An endoscopy support method comprising:
acquiring an endoscopic image captured by an endoscope;
acquiring identification information of a patient;
estimating a position of the endoscope in a large intestine;
retrieving previous examination information related to the acquired identification information and the estimated position of the endoscope from a storage means for storing previous examination information associating the identification information of the patient, a position where a lesion was detected in a previous examination, an image of the lesion, and information regarding the lesion; and
displaying the previous examination information on a display device in a case where the retrieved previous examination information includes the information regarding the lesion.

### (Supplementary Note 9)

A recording medium storing a program that causes a computer to execute processing, the processing comprising:
acquiring an endoscopic image captured by an endoscope;
acquiring identification information of a patient;
estimating a position of the endoscope in a large intestine;
retrieving previous examination information related to the acquired identification information and the estimated position of the endoscope from a storage means for storing previous examination information associating the identification information of the patient, a position where a lesion was detected in a previous examination, an image of the lesion, and information regarding the lesion; and
displaying the previous examination information on a display device in a case where the retrieved previous examination information includes the information regarding the lesion.

While the present disclosure has been particularly shown and described with reference to example embodiments and examples thereof, the present disclosure is not limited to these example embodiments and examples. It will be understood by those of ordinary skill in the art that various changes in form and details may be made therein without departing from the spirit and scope of the present disclosure as defined by the claims.

### DESCRIPTION OF SYMBOLS

1 endoscopy support device
2 display device
3 endoscopic scope
11 processor
12 memory
17 database (DB)
100 endoscopy system
111 patient ID acquisition unit
112 endoscopic video acquisition unit
113 position determination unit
114 previous examination information retrieval unit
115 display data generation unit
116 inflammation presence/absence determination unit
117 examination information acquisition unit

What is claimed is:

1. An endoscopy support device comprising:
at least one memory configured to store instructions; and
at least one processor configured to execute the instructions to:
acquire an endoscopic image captured by an endoscope;
an acquire identification information of a patient;
estimate a position of the endoscope in a large intestine;
store, in a storage, previous examination information that associates the identification information of the patient, a position where a lesion was detected in a previous examination, an image of the lesion, and information regarding the lesion;
retrieve previous examination information at the position of the endoscope from the storage based on the acquired identification information and the estimated position of the endoscope; and
display the previous examination information on a display device in a case where the retrieved previous examination information includes the information regarding the lesion.

2. The endoscopy support device according to claim 1, wherein the one or more processors are further configured to determine whether the large intestine is in an inflammatory state,
wherein the one or more processors display information indicating the position of the endoscope and presence or absence of inflammation at the position of the endoscope on a model image showing an entire large intestine.

3. The endoscopy support device according to claim 2, wherein
the previous examination information includes information associating the identification information of the patient, a position where inflammation was detected in the previous examination, and an image of the inflammation, and
the one or more processors display the previous examination information on the display device in a case where the retrieved previous examination information includes the image of the inflammation.

4. The endoscopy support device according to claim 3, wherein
the one or more processors retrieve the previous examination information based on the identification information of the patient,
the one or more processors generate a model image showing an entire large intestine in such a way as to be capable of identifying a position where previous inflammation was detected and a position where the previous inflammation was not detected, based on the previous examination information, and
the one or more processors display information indicating a current position of the endoscope and presence or absence of inflammation at the position of the endoscope on the model image indicating the entire large intestine.

5. The endoscopy support device according to claim 4, wherein
the one or more processors determine a progression state of the inflammation based on a result of the inflammation presence or absence determination and the previous examination information, and
the one or more processors display the progression state of the inflammation on the display device.

6. The endoscopy support device according to claim 1, wherein the one or more processors estimate the position of the endoscope in the large intestine from the endoscopic image.

7. The endoscopy support device according to claim 1, wherein the one or more processors estimate the position of the endoscope in the large intestine from a magnetic coil incorporated in the endoscope.

8. An endoscopy support method comprising:
acquiring an endoscopic image captured by an endoscope;
acquiring identification information of a patient;
estimating a position of the endoscope in a large intestine;
retrieving previous examination information related to the acquired identification information and the estimated position of the endoscope from a storage for storing previous examination information associating the identification information of the patient, a position where a lesion was detected in a previous examination, an image of the lesion, and information regarding the lesion; and
displaying the previous examination information on a display device in a case where the retrieved previous examination information includes the information regarding the lesion.

9. A non-transitory computer readable recording medium storing a program that causes a computer to execute processing, the processing comprising:
acquiring an endoscopic image captured by an endoscope;
acquiring identification information of a patient;
estimating a position of the endoscope in a large intestine;
retrieving previous examination information related to the acquired identification information and the estimated position of the endoscope from a storage for storing previous examination information associating the identification information of the patient, a position where a lesion was detected in a previous examination, an image of the lesion, and information regarding the lesion; and
displaying the previous examination information on a display device in a case where the retrieved previous examination information includes the information regarding the lesion.

10. The endoscopy support device according to claim 2, wherein the one or more processors are further configured to:
employ a machine learning model for inflammation detection and provide decision making assistance to physicians by highlighting areas of concern.
